(19) United States
(12) Patent Application Publication
(10) Pub. No.: US 2008/0268491 A1
(43) Pub. Date: Oct. 30, 2008

(54) INSTRUMENTS FOR DIRECT DETECTION OF FREE METALS IN FLUIDS AND METHODS TO DIAGNOSE METAL-RELATED DISEASES AND DETERMINE PHARMACOLOGIC DOSING REGIMENS

(75) Inventors: Steve H. Kanzer, Ann Arbor, MI (US); John S. Althaus, Ann Arbor, MI (US)

Correspondence Address: GARVEY SMITH NEHRBASS & NORTH, LLC, LAKEWAY 3, SUITE 3290, 3838 NORTH CAUSEWAY BLVD., METAIRIE, LA, 70002, US

(73) Assignee: PIPEX, INC., Ann Arbor, MI (US)

(21) Appl. No.: 12/033,849

(22) Filed: Feb. 19, 2008

Related U.S. Application Data
(60) Provisional application No. 61/024,063, filed on Jan. 28, 2008.
(60) Provisional application No. 60/890,491, filed on Feb. 18, 2007.

(51) Int. Cl. G01N 33/49; C12M 1/34

(52) U.S. Cl. 435/29; 435/287.1

(57) ABSTRACT

A method and apparatus for measuring the level of metal in a biological sample can employ a current measuring device. It preferably includes a display for displaying the level of metal, and preferably free metal, in the sample. It can use a test strip interfaced to a potentiostat. The test strip preferably includes layers that separate a part of the sample which contains the free metal. Electrodes enable measurement of free metal in the separated part of the sample.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A “MICROFICHE APPENDIX”

Not applicable

### BACKGROUND OF THE INVENTION

### General Background of the Invention

A number of diseases and health conditions have been linked to lower or higher serum metal levels, such as copper, and iron Convenient direct measurement of free and bound copper and iron in body fluids is a significant medical need. Measurement of copper and iron provides functions that are diagnostic, prognosticative and/or maintenance-serving.

Prior to the present invention, currently available methods of estimating free and bound copper and iron in plasma and serum rely upon estimations that are subject to a considerable amount of variability and inaccuracy. For example, the current “gold standard” for measuring “free scrum copper” involves a measurement of total serum copper (generally determined by flame absorbance spectroscopy) and subtraction of the estimated amount of copper theoretically bound to the serum protein ceruloplasmin. Such estimation, however, can be highly inaccurate, due to the variation in actual copper-ceruloplasmin binding that varies between individuals, as well as factors, such as aging, during a person's lifetime. For example, it is widely assumed that a single ceruloplasmin protein binds seven copper atoms. However, with aging the binding capacity of ceruloplasmin can be as low as five copper atoms.

Since an elevated pool of free copper in serum can be toxic to the central nervous system and other organs, what is needed, and what the present invention provides, is an instrument and methodology capable of directly measuring free and bound copper without relying on estimations that may or may not be correct. A preferred embodiment of the present invention also provides a method of diagnosing persons having potentially toxic elevated free copper pools as well as means of dosing one or more copper lowering agents based upon direct measurements of free serum copper pools. Examples of diseases that may be associated with elevated free serum copper pools include Wilson's disease, Alzheimer's disease, Parkinson's disease, schizophrenia, atherosclerosis, diabetes, and other common diseases.

Wilson's disease is a rare autosomal recessive inherited disorder of copper metabolism

The condition is characterized by excessive deposition of copper in the liver, brain, and other tissues. The major physiologic aberration is decreased excretion of copper by the liver. The genetic defect, localized to chromosome arm 13q, has been shown to affect the copper-transporting adenosine triphosphatase (ATPase) gene (ATP7B) in the liver. Patients with Wilson's disease usually present with liver disease during the first decade of life subsequent to neuropsychiatric illness during the second and third decade. The diagnosis is confirmed by measurement of serum ceruloplasmin, urinary copper excretion, serum free copper and hepatic copper content, as well as the detection of Kayser-Fleischer rings.

Menkes Disease is caused by a defective gene that regulates the metabolism of copper in the body. Because it is an X-linked gene, the disease primarily affects male infants. Copper accumulates at abnormally low levels in the liver and brain, but at higher than normal levels in the kidney and intestinal lining. Affected infants may be born prematurely. Symptoms appear during infancy. Normal or slightly slowed development may proceed for two to three months, and then there will be severe developmental delay and a loss of early developmental skills. Menkes Disease is also characterized by seizures, failure to thrive, subnormal body temperature, and strikingly peculiar hair, which is kinky, colorless or steel-colored, and easily broken. There can be extensive neurodegeneration in the gray matter of the brain. Arteries in the brain can also be twisted with frayed and split inner walls. This can lead to rupture or blockage of the arteries. Weakened bones (osteoporosis) may result in fractures.

The relation between serum free copper and Alzheimer's Disease has received considerable attention over the last few years. Copper is an essential element and under normal physiologic circumstances is maintained complexed to proteins. This appears to be a protective mechanism developed in mammals to prevent “free copper” availability.

In the free form copper is a highly reactive metal causing free radical formation and oxidation. Normally, most copper is bound to ceruloplasmin (Cp) in the serum. In the brain, protective mechanisms have also evolved to keep copper complexed to intracellular proteins and those in the cerebrospinal fluid (CSF). Copper has been shown to be bound to the Tau protein, amyloid precursor protein, the beta secreatase, beta amyloid protein, and apoE. It is believed by the present inventors that copper is normally bound to these protein as a protective mechanism against excess copper. Indeed, in vitro studies have shown that amyloid precursor protein expression is down regulated in copper depleted cells. The presence of beta amyloid plaques and intracellular neurofibullary tangles appear to result from copper binding to beta-amyloid and the tau protein, respectively, and induction of protein structural changes.

This appears to be a pathological mechanism to deal with excess free copper. Thus, under conditions of excess free copper in the brain, these protein structural changes appear to be the markers of the disease and not the cause. It should be noted that in the elderly, two conditions lead to elevated copper in the brain. First, the blood brain barrier become more permeable as we age and thereby allows exchange between the serum and the CSF to occur more easily. Secondly, as liver function diminishes, the amount of copper associated with Cp decreases, from about 7 copper atoms/Cp molecule to about 5 copper atoms/Cp molecule, making free copper more available for transport to the brain.

Interestingly, the work of Squitti and associates have shown that the level of copper unassociated with ceruloplasmin is markedly elevated in AD subjects compared to age-matched controls.

In addition, the work of Sparks et al., Proc Natl Acad Sci USA, 2003 Sep. 16; 100(19):11065-9; Epub 2003 Aug. 14, has shown a direct link between copper in the drinking water in an experimental rabbit model of cholesterol-diet induced Alzheimer's disease. In this work these investigators demonstrated that animals consuming distilled water had markedly reduced AD plaques in the brain compared to tap water controls. Furthermore, they determined that copper was the culprit mineral in the water that induced this effect. The cholesterol-diets likely caused endothelial damage to the blood brain barrier allowing the easy penetration to the brain compartment. The tap water treated rabbits also suffered dramatically poorer memories in complex tests.

### Amyotrophic Lateral Sclerosis (ALS):

Goto J J, Zhu H, Sanchez R J, Nersissian A, Gralla E B, Valentine J S, Cabelli D E. Loss of in vitro metal ion binding specificity in mutant copper-zinc superoxide dismutases associated with familial amyotrophic lateral sclerosis. J Biol Chem. 2000 Jan. 14; 275(2): 1007-14.

The presence of the copper ion at the active site of human wild type copper-zinc superoxide dismutase (CuZnSOD) is essential to its ability to catalyze the disproportionation of superoxide into dioxygen and hydrogen peroxide. Wild type CuZnSOD and several of the mutants associated with familial amyotrophic lateral sclerosis (FALS) (Ala(4)-->Val, Gly(93)-->Ala, and Leu(38)-->Val) were expressed in Saccharomyces cerevisiae. Purified metal-free (apoproteins) and various remetallated derivatives were analyzed by metal titrations monitored by UV-visible spectroscopy, histidine modification studies using diethylpyrocarbonate, and enzymatic activity measurements using pulse radiolysis. From these studies it was concluded that the FALS mutant CuZnSOD apoproteins, in direct contrast to the human wild type apoprotein, had lost their ability to partition and bind copper and zinc ions in their proper locations in vitro. Similar studies of the wild type and FALS mutant CuZnSOD holoenzymes in the “as isolated” metallation state showed abnormally low copper-to-zinc ratios, although all of the copper acquired was located at the native copper binding sites. Thus, the copper ions are properly directed to their native binding sites in vivo, presumably as a result of the action of the yeast copper chaperone Lys7p (yeast CCS). The loss of metal ion binding specificity of FALS mutant CuZnSODs in vitro may be related to their role in ALS.
Forsleff L, Schauss A G, Bier I D, et al. Evidence of functional zinc deficiency in Parkinson's disease. J Altern Complement Med 1999; 5:57-64.
Uitti R J, Rajput A H, Rozdilsky B, et al. Regional metal concentrations in Parkinson's disease, other chronic neurological diseases, and control brains. Can J Neurol Sci 1989; 16:310-4.
Pall H S, Williams A C, Blake D R, et al. Raised cerebrospinal fluid copper concentration in Parkinson's disease. Lancet 1987; 2(8553):238-41.
Dexter D T, Carayon A, Javoy-Agid F, et al. Alterations in the levels of iron, ferritin and other trace metals in Parkinson's disease and other neurodegenerative diseases affecting the basal ganglia. Brain 1991; 14:1953-75.

Copper levels were significantly higher in the cerebrospinal fluid of patients with idiopathic Parkinson's disease than in the control group (Pall et al. 1987). Although the specific reason for elevated copper levels was not known, copper is generally high when there is chronic inflammation, such as that caused by autoimmune or undetected allergy reactions. Furthermore, a copper enzyme is required to convert tyrosine into levodopa. Therefore, elevated levels of brain copper may be an attempt to stimulate production of levodopa. However, high copper levels in the presence of antioxidant deficiencies tend to cause increased free-radical damage to nerve cell DNA.

People with Parkinson's disease have shown both decreased and increased levels of zinc and copper. Both nutrients function in the antioxidant enzyme superoxide dismutase (SOD). SOD tends to be low in the area of the brain involved in Parkinson's disease. In theory, therefore, low levels of zinc and copper could leave the brain susceptible to free radical damage. However, copper and zinc (as well as iron) taken in excess can also act as pro-oxidants, and all have been associated with an increased risk of developing Parkinson's disease in preliminary research. Insufficient evidence currently exists for either recommending or avoiding supplementation with zinc and copper.

### Other Diseases:

Gray hair and skin wrinkles are signs of copper deficiency. Other diseases involving copper deficiency include: anemia, baldness, benign prostatic hyperplasia, bone and joint abnormalities, brain disturbances, diarrhea, elevated LDL cholesterol levels, general weakness, hypoglycemia, impaired immune function, impaired respiratory function, osteoporosis, retinal degeneration, rheumatoid arthritis and skin sores.

Imaging technologies including CT scan, PET scan, MRI, ultrasound and nuclear medicine of various organs have been used to detect disease in patients exhibiting copper abnormalities.

The diagnosis of copper abnormalities is typically made by a blood test for serum ceruloplasmin, a liver biopsy for measurement of liver copper content and a test for urinary copper-excretion levels.

Analytical methods for measuring copper in biological and environmental samples include atomic absorption spectrometry, anodic stripping voltammetry, graphite furnace atomic absorption, inductively coupled plasma-atomic emission spectroscopy and inductively coupled plasma-mass spectrometry.

Detection of copper in biological samples for purposes of disease diagnosis is complicated by the fact that copper exists in a variety of discrete and separate pools. In plasma, copper is bound to ceruloplasmin (Log K=˜12), albumin (Log K=˜7), transcuprein (Log K=<7), amino acids (Log K=7 to 5) and to a lesser extent other ligands. In addition, a very small percentage of copper exists free in solution.

Diagnosis of copper-dependent pathologies depends on the distribution of copper within these pools. As such copper can be described as good and bad. Good copper is tightly bound to structures in which the Log K is 12 or greater. Bad copper is loosely bound to structures in which the Log K is 7 or less. Copper is bad when it is loosely bound and available to participate in free radical reactions.

Total copper in biological samples is typically detected using atomic absorption spectrometry in which discernment between pools of copper is not possible. Alternatively, methods which resolve serum into protein and non-protein fractions have been employed. Unfortunately these methods do not discriminate between copper bound to ceruloplasmin, albumin and transcuprein. In this case, protein-bound copper still contain both good and bad copper and the usefulness of this measurement falls into question.

The present invention uses a potentiostat to measure free levels of copper and other metals in bodily fluids. Other techniques to “directly” detect free and bound metal levels which might be applied include, for example, ultrafiltration, immunometric, separation column (i.e. Sephrose), magnetic bead, immobilized metal affinity chromatography, 2D gel electrophersis (i.e. SDS-PAGE) and other protein separation techniques available to those skilled in the art. In these techniques, metal binding proteins of interest, such as ceruloplasmin and transferrin, may be separated provided the methodologies are not too denaturing, and the level of metals of interest bound to such metalloproteins of interest can be subsequently determined by flame absorbance spectroscopy, for example.

This bound metal measurement can then be subtracted from the level of total metal of interest in the original fluid sample to arrive at an estimate of the free metal pool of interest in such a sample. Such techniques are, however, labor intensive, time consuming, subject to inaccuracies due to metal stripping effects such processes may have on the samples and still rely upon a subtraction estimate to arrive at an estimate of the free metal levels of interest in the fluid sample (such as non-ceruloplasmin bound copper and nontransferrin bound iron (NTBI), as opposed to directly measuring the actual and kinetic availability of the metal of interest in the original sample.

Elevated free metal levels in serum samples can be of interest in both the diagnosis and proper treatment of a number of diseases, such as Wilson's disease, iron overload related to blood transfusions, neurodegenerative and CNS diseases, such as Alzheimer's disease, Parkinson's disease, ALS, Pick's disease, prion-related disease, schizophrenia, diabetes, heart disease and atherosclerosis, for example. The body fluid sample need not be limited to serum, but may also comprise, blood, plasma, cerebrospinal fluid, urine, tears, and saliva, for example.

Elevated free metal levels such as copper and iron can be treated with pharmacologic agents that can either complex such metals, such as thiomolybates and thiotungstates in the case of copper, and Exjade in the case of iron, or agents that chelate metals such as penicillamine, trientine, clioquinol, EDTA and desferroxamine, for example, or by agents that block absorption of metals, such as zinc acetate.

Incorporated herein by reference are the following patent publications listing the present inventors are inventors: WO 2007/092966 A2, WO 2007/092966 A3, and US2007/209950 A1.

### BRIEF SUMMARY OF THE INVENTION

In a preferred embodiment, the present invention can also be used to determine and titrate the appropriate dose and course of treatment of a pharmacologic agent that either complexes, chelates or blocks the absorption of metals of interest. Since certain metals, such as copper and iron, are essential trace metals, it has heretofore been difficult to determine the appropriate dose and course of treatment with agents that complex, chelate or block the absorption of such metals for example. (Indeed, in certain instances supplementation with such metals may even be the best treatment course). As a result, chelation therapy as currently practiced by the art is all too often associated with instances of metal deficiency, which can manifest as leucopenia and anemia, for example.

Patients undergoing chelation therapy require careful monitoring by their treating physicians and even under the best supervision instances of metal deficiency are still common. Such instances are due to the fact that, until the present invention, treating and prescribing physicians lacked an accurate and reliable (and preferably rapid as in point-of-care) method to directly measure free and otherwise available metal levels in patients requiring treatment. Instead, they rely on a process of trial and error and careful supervision which results in cases of essential metal deficiency and is time consuming and burdensome on the physicians, patients, laboratories and the healthcare system. Accordingly, it is an object of the present invention to provide a method of determining an initial, adjusted and/or titrated dosage amount and regimen of an anti-metal agent that is based upon a direct measurement of a metal of interest. It is anticipated by the present inventors that such direct metal measurement methodology can be incorporated into a package insert and prescribing information of an anti-metal agent to assist treating physicians and others in selecting, adjusting and titrating the appropriate dose, regimen and course of treatment of an anti-metal therapy so as to maintain an appropriate therapeutic range or index. Such methodology may involve calculations based upon other parameters such as body weight, free and total metal levels, as well as extrapolations determined by clinical trials or clinical experience.

This application may be suited to a number of diseases in which measurement of free copper in serum can be diagnostic. Most notable is Wilson's disease. Wilson's is a genetic disease in which pathologic copper accumulation affects the health and function of the liver. In Wilson's, total serum copper can be normal or slightly decreased from normal. Total serum copper falls into pools that are bound or free. Most of the bound copper is associated with ceruloplasmin (Cp) which in normal subjects represents about 80% of total copper. The free copper pool is distributed between albumin, peptides and amino acids. In Wilson's, ceruloplasmin can be decreased but it is the free copper that is increased and believed to play a diagnostic as well as a pathologic role.

### BRIEF DESCRIPTION OF THE SEVERAL VIEWS OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is a partial perspective view of the preferred embodiment of the apparatus of the present invention;

FIG. 2 is a partial schematic view of the preferred embodiment of the apparatus of the present invention showing the relationship of a potentiostat to the electrode used to measure free metal;

FIGS. 3A-3C are graphs showing electrochemical detection of copper sulfate;

FIG. 4 is a graph illustrating SWV measurement of copper;

FIG. 5 is a graph illustrating SWV measurement of copper;

FIG. 6 is a graph showing electrochemical detection of copper as copper sulfate and in serum after 3 and 30 minutes of deposition;

FIG. 7 is a graphical analysis of free copper in serum from Wilson's patients;

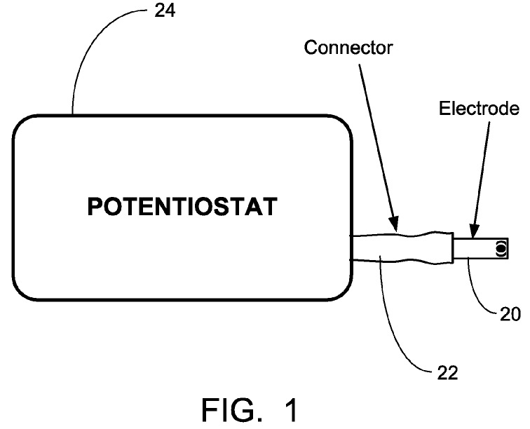

### DETAILED DESCRIPTION OF THE INVENTION

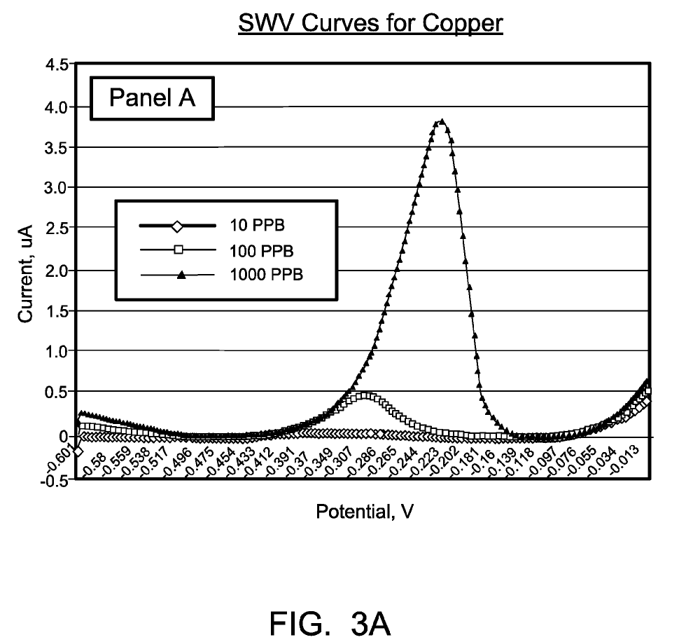

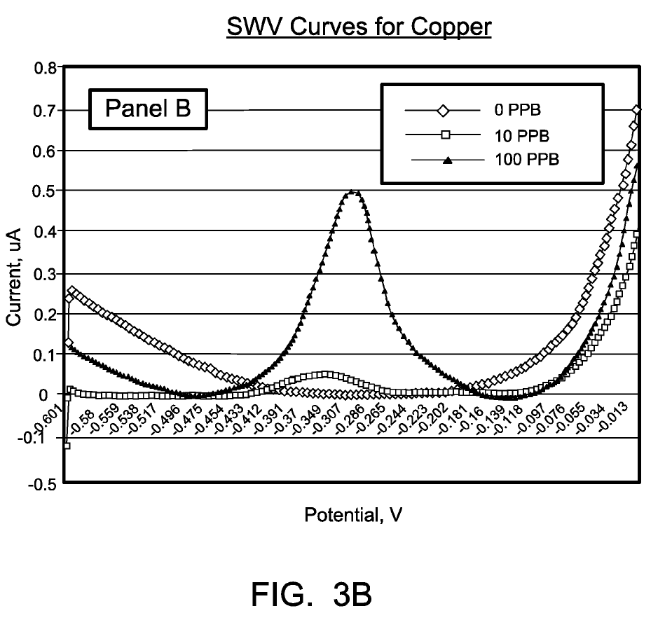

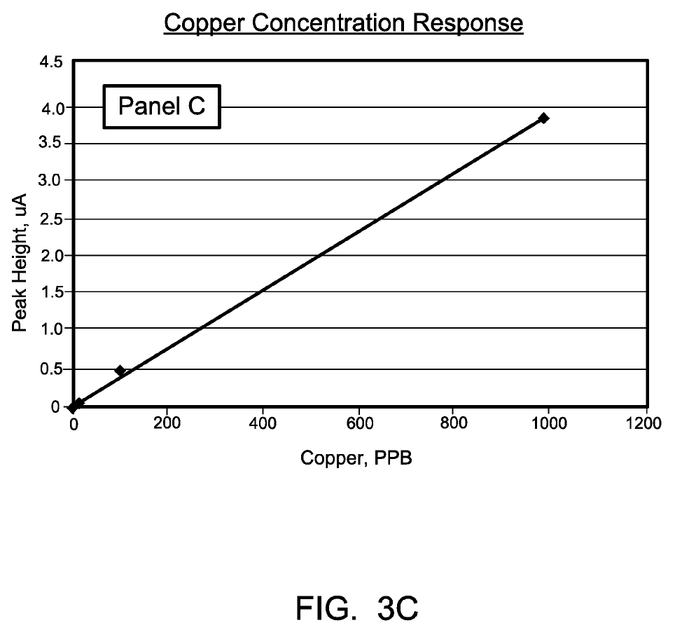

FIGS. 3A, 3B and 3C illustrate electrochemical detection of copper sulfate. FIGS. 3A, 3B and 3C show SWV (square wave parameters) curves for copper at 0, 10, 100 and 1000 PPB (parts per billion). Within this range, the data gives a linear concentration response range.

In FIG. 3A, square wave voltammetry curves of copper sulfate at 10 PPB, 100 PPB and 1000 PPB are shown. In FIG. 3B, square wave voltammetry curves of copper sulfate at 0 PPB, 10 PPB and 100 PPB are shown. FIG. 3C shows a copper concentration response curve of data shown in FIGS. 3A and 3B.

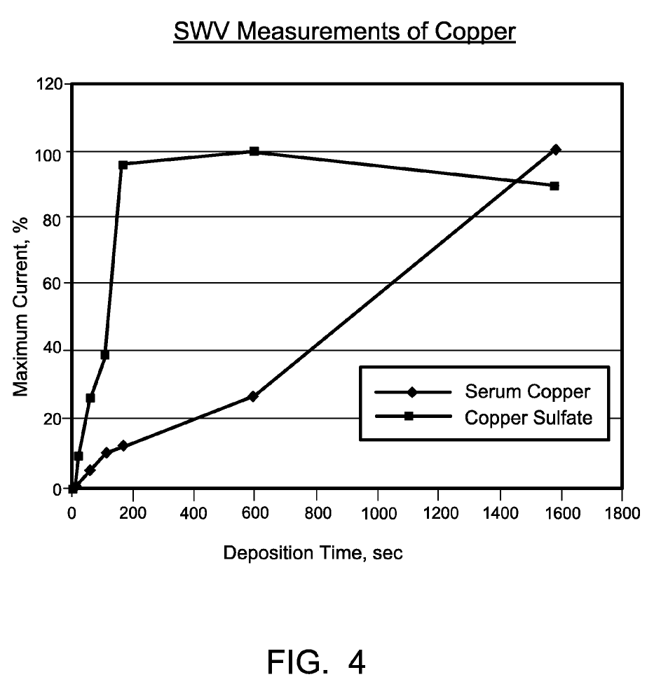

In FIG. 4, SWV (square wave voltammetry parameters) copper peak heights (as a percent of the largest peak height within each time course) were plotted in terms of deposition time (seconds). The line plot with squares represents data for copper sulfate and the line plot with diamonds with diamonds represents data for serum copper. In FIG. 4, for copper sulfate, approximate peak height saturation was achieved by around 3 minutes of deposition time.

The diamonds represent copper measured from serum (1 to 10 dilution in 0.1 N HCl). The squares represent copper measured as copper sulfate (100 PPB in 0.1 N HCl). The maximum peak current height for 100 PPB copper sulfate was about 1.3 uA. The maximum peak current height for copper serum was about 1 uA.

The data shown in FIG. 4 demonstrates that: copper sulfate shows a monophasic response; serum copper shows a multiphasic response; the free copper response is over by 200 seconds;

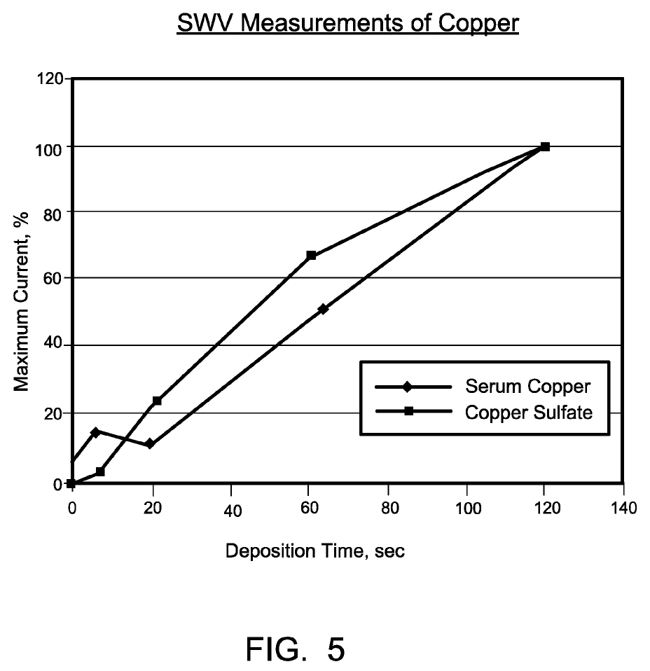

In FIG. 5, the diamonds represent copper measured from serum (1 to 10 dilution in 0.1 N HCl). The squares represent copper measured as copper sulfate (100 PPB in 0.1 N HCl). Copper measured from serum is in lines with diamonds. Copper measured as copper sulfate is in lines with squares. The initial rate for measurement of copper sulfate is superimposable with the initial rate for measurement of serum copper.

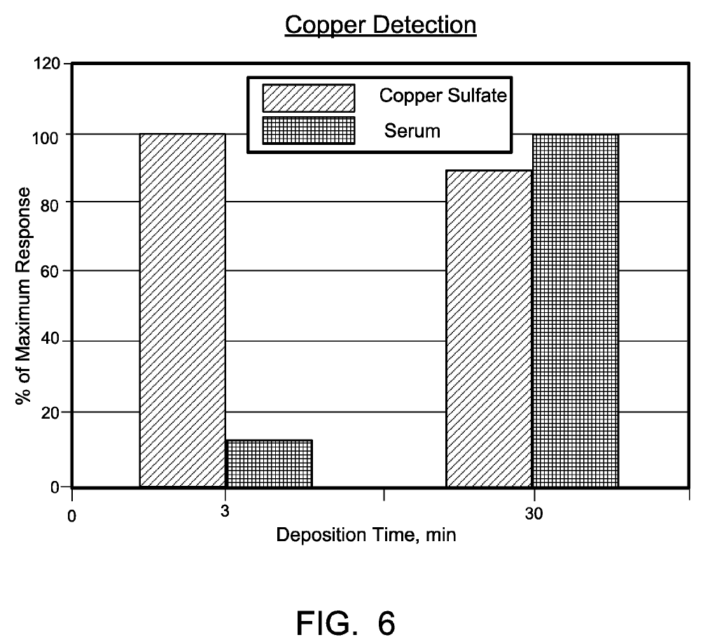

In FIG. 6, electrochemical detection of copper as copper sulfate and in serum are shown with different cross hatch patterns, after 3 and 30 minutes of deposition.

Copper measured as copper sulfate was 100 PPB in 0.1 N HCl. Copper measured from serum was 1 to 10 dilution in 0.1 N HCl. The maximum peak current height for 100 PPB copper sulfate was about 1.3 uA. The maximum peak current height for copper serum was about 1 uA.

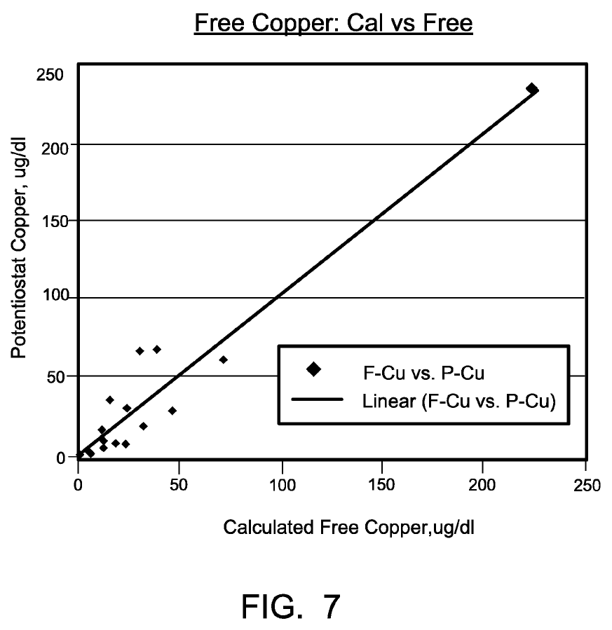

FIG. 7 shows an analysis of free copper in serum from Wilson's patients. Serum samples from Wilson's patients were provided. Free copper was determined in two ways. One way was based on a calculation measuring total copper by atomic absorption and subtracting the bound copper based on a measurement of ceruloplasmin in each sample (X-axis). The other way (Y-axis) was based on the measurement of free copper using a potentiostat by the method described under Experimental (below). The correlation coefficient describing the linear relationship between both sets of data is r=0.96.

### EXPERIMENTAL

### Introduction

Squitti and researchers have demonstrated that “Free Copper” tracks with the MMSE in Alzheimer's. Historically free copper has been measured using methods that are less than direct. The method of the present invention provides a potentiometric method for directly measuring free copper concentrations in serum.

If the time course of copper deposition of copper sulfate on a carbon working electrode can be determined, then this time course should pattern the deposition of free copper in serum.

### Method:

1. The following Square Wave Parameters (SWV method) were used:

| | |
|---|---|
| E begin: −0.6 V | |
| E end: −0.0 V | |
| E step potential: 0.003 V | |
| E amplitude: 0.028 V | |
| Freq: 15 Hz | |
| E cond: −0.200 V | t cond: 60 s |
| E dep: −2 V | t dep: 0 to 1600 s |
| E eq: −0.150 | t eq: 30 s |

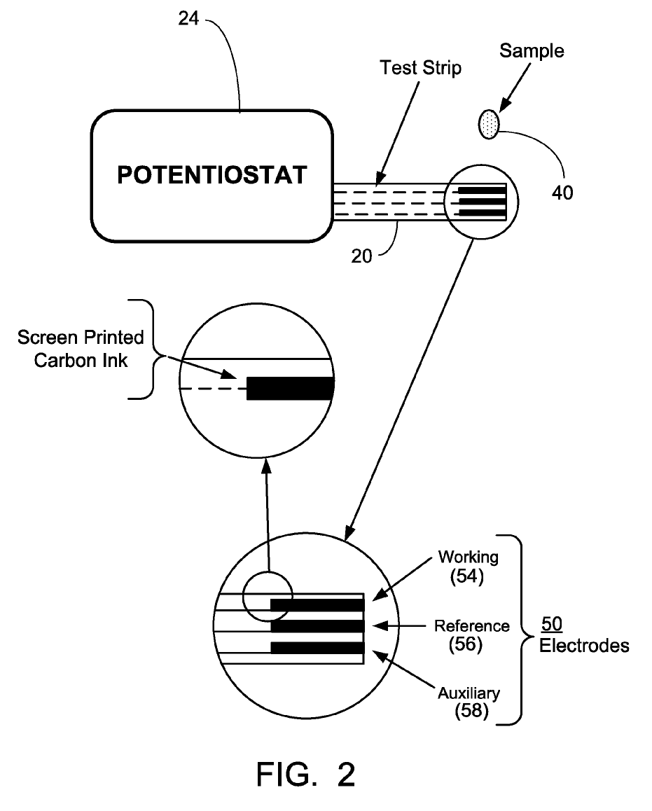

2. A PalmSens™ potentiostat was used.
3. University of Florence heavy metal electrodes 50 (see FIGS. 2, 10, 12) were used. These electrodes 50 consisted of carbon, silver and carbon for the working 54, reference 56 and counter 58 (or auxiliary) electrodes, respectively.
4. In FIG. 4, copper sulfate was measured as a 100 PPB solution in 0.1 N HCl.
5. Pathogen-free human serum was purchased from Sigma-Aldrich.
6. Solutions containing 100 ul of plasma and 900 ul of 0.1 N HCl were prepared fresh just prior to its application to the electrode.
7. Following every SWV measurement, electrodes were cleaned by applying a 100 ul solution of 0.1 N HCl to the electrode using the SWV method in which a deposition potential of 2 V for 60 seconds was used.
8. The SWV method was used after the application of either 100 ul of copper sulfate or 100 ul of serum solution.
9. A deposition potential of −2V was applied for either 3 or 30 minutes.
10. A copper SWV signal was observed at about −0.3 volts.
11. The peak height for each copper curve was measured using commercially available PalmSens™ software.
   The results are shown in FIGS. 3A, 3B, 3C and 4.

Detection of copper as copper sulfate peaked at around 3 minutes. Detection of copper in serum peaked at around 30 minutes. The amount of copper detected in serum at 3 minutes was only about 13% of the copper detected at 30 minutes. Assuming that total serum copper has been determined by 30 minutes, then the free copper (defined by data at 3 minutes) represents about 13% of the total copper. This value seems reasonable based on other published reports of free copper in serum (20% plus or minus).

The following is a list of parts and materials suitable for use in the present invention.

PARTS LIST

| Part Number | Description (preferred materials) |
|---|---|
| 20 | test strip element |
| 24 | potentiostat |
| 50 | electrodes |
| 54 | working electrode |
| 56 | reference electrode |
| 58 | counter/auxiliary electrode |

The foregoing embodiments are presented by way of example only; the scope of the present invention is to be limited only by the following claims.

1-212. (canceled)

213. Apparatus for measuring free copper levels in blood, comprising:
a multifunctional filter for creating filtrate from a blood sample by filtering out particles which would interfere with measurement of free copper in the blood sample and conditioning the filtrate to an appropriate pH to allow current flow therethrough, the particles including particles larger than about 130 kD and particles having bound copper bound thereto;
a detector apparatus including electrodes for detecting current flow through the filtrate;
a display for displaying the free copper level in the blood sample based on the current flow detected between the electrodes.

214-251. (canceled)

252. A method of detecting a metal in blood serum, comprising the steps of:
a) applying blood to a first layer;
b) contacting the first layer with a second layer;
c) transferring the blood from the first layer to a third layer using the transfer layer;
d) separating serum from blood cells with the third layer;
e) capturing the serum of step “d” on a capture pad; and
f) analyzing the pad of step “e” for the metal.

253. The method of claim 252 wherein the metal is copper.

254. The method of claim 252 wherein the metal is zinc.

255. The method of claim 252 wherein at least one of the layers is a membrane.

256. The method of claim 252 wherein multiple of the layers are membranes.

257. The method of claim 252 wherein in step “e” the serum is removed for analyte processing.

258. The method of claim 252 wherein in step “d” the third layer separates serum from leucocytes.

259. The method of claim 252 wherein in step “d” the third layer separates serum from erythrocytes.

260. The method of claim 252 wherein in step “d” the third layer separates serum from platelets.

261. The method of claim 252 wherein the second layer is a transfer membrane.

262. The method of claim 252 further comprising adding to the first layer a reagent that enables the flow of blood through the first layer.

263. The method of claim 250 further comprising at least partially drying the pad of step “e” after step “d”.

264-266. (canceled)

267. An instrument that measures levels of free metal in a biological sample, comprising:
a) a potentiostat;
b) a test strip interfaced to the potentiostat, the test strip including layers that separate serum from a biological sample; and
c) electrodes that enable measurement of free metal in the separated serum.

268. The instrument of claim 267 wherein the free metal is selected from the group consisting of copper, iron, zinc and lead.

269-273. (canceled)
